(19) United States
(12) Patent Application Publication
(10) Pub. No.: US 2019/0328752 A1
(43) Pub. Date: Oct. 31, 2019

(54) COMPOUNDS FOR THE TREATMENT OF NIEMANN-PICK DISEASE TYPE C1

(71) Applicants: UNIVERSITEIT MAASTRICHT, Maastricht (NL); ACADEMISCH ZIEKENHUUIS MAASTRICHT, Maastricht (NL)

(72) Inventors: Tom Houben, Maastricht (NL); Jogchum Plat, Geulle (NL); Ronit Sverdlov, Maastricht (NL)

(21) Appl. No.: 16/087,883

(22) PCT Filed: Mar. 23, 2017
(86) PCT No.: PCT/EP2017/056955

§ 371 Date: Sep. 24, 2018

(30) Foreign Application Priority Data
Mar. 25, 2016 (EP) 16162475.4

(51) Int. Cl. A61K 31/575; A61P 1/16

(52) U.S. Cl. CPC A61K 31/575; A61P 1/16

(57) ABSTRACT

The invention is in the field of medical treatments. The invention provides means and methods for the treatment of Niemann-Pick disease type C1. More specifically, it provides a composition comprising a plant sterol or plant stanol for use in the treatment of Niemann-Pick disease type C1. Preferred embodiments of the invention concern treatments with a plant stanol which is a chemically saturated plant sterol, or wherein the plant stanol is esterified with a fatty acid to form a fatty acid ester.

### FIELD OF THE INVENTION

The invention is in the field of medical treatments. The invention in particular addresses the treatment of Niemann-Pick disease type C1.

### BACKGROUND OF THE INVENTION

Although rare, Niemann-Pick disease type C1 (NPC1) is an extremely severe disease with the majority of patients dying between 10 and 25 years of age (Vanier MT. Niemann-Pick disease type C. Orphanet journal of rare diseases. 2010; 5:16). Moreover, clinical features of NPC are extremely heterogeneous and range from systemic (lung, spleen, lung) to neurological symptoms.

NPC1 is an inherited lysosomal lipid storage disease resulting from a deletion in the NPC1 gene, leading to impaired intracellular lipid transport and accumulation of unesterified cholesterol in lysosomes of various tissues (Parkinson-Lawrence E J, Shandala T, Prodoehl M, Plew R, Borlace G N, Brooks D A. Lysosomal storage disease: revealing lysosomal function and physiology. Physiology (Bethesda). 2010; 25(2):102-15). The NPC1 gene encodes a lysosomal membrane protein involved in the translocation of cholesterol from the lysosome to the cytoplasm.

One of the proposed mechanisms that contribute to NPC1 pathology is an inappropriate inflammatory response, mediated by dysregulated activation of macrophages (Maxfield FR, Tabas I. Role of cholesterol and lipid organization in disease. Nature. 2005; 438(7068):612-21.). Cholesterol trapped in lysosomes of macrophages has been shown to be very resistant to mobilization into the cytoplasm.

Currently, therapeutic options for NPC disease are limited. The substrate reducer Miglustat, which diminishes the accumulation of the toxic GM2 and GM3 gangliosides by inhibiting glucosylamide synthase, has been approved in Europe, Canada and Japan for the treatment for the neurological manifestations in adult and pediatric NPC disease patients (1).

Treatment strategies both targeting the visceral and neurological manifestations of NPC disease are not available.

Another approach is the use of chemical chaperones which are able to enhance the folding of either the mutant NPC1 or NPC2 protein thereby facilitating the cholesterol efflux from the late endosomes/lysosomes.

Furthermore, cyclodextrin, a membrane-impermeable cyclic oligosaccharide, has been shown to replace the function of NPC1 and NPC2 and promote cholesterol esterification by Acetyl-Coenzyme A acetyltransferase (ACAT) within the late endosomes/lysosomes (2, 3). However, the inability of cyclodextrin to cross the blood brain barrier underlines the ineffectiveness of cyclodextrin as a treatment for NPC disease (4).

In conclusion, NPC disease is a heterogenous neurovisceral disease characterized by lipid accumulation within the late endosomes/lysosomes and the presence of foamy lipid laden macrophages. Currently, Miglustat is the only effective treatment for the reduction of pathology in NPC disease. This underlines the urgent need to develop effective treatment strategies which target both visceral and neurological manifestations.

### SUMMARY OF THE INVENTION

The invention relates to a composition comprising a plant sterol or plant stanol for use in the treatment of Niemann-Pick disease type C1. This may also be worded as a method for treating Niemann-Pick disease type C1, wherein a composition comprising a plant sterol or plant stanol is administered to a subject in need of such a treatment. In yet other words, the invention relates to the use of a plant sterol or plant stanol for the manufacture of a medicament for the treatment of Niemann-Pick disease type C1.

### DETAILED DESCRIPTION OF THE INVENTION

To investigate whether plant stanols could reduce inflammation in vitro, bone marrow-derived macrophages (BMDMs) of NPC1 wild type (control) or mutant mice were used. In vitro, NPC1 WT and mutant BMDMs were stimulated with oxLDL and lipopolysaccharide (LPS) to induce lysosomal cholesterol accumulation and an inflammatory response, respectively. To determine whether plant stanols were able to reduce inflammation in NPC1 disease-like model in vivo, NPC1 WT and mutant BMDMs were stimulated with cyclodextrin (carrier control) or sitostanol (0.6 μM). In vivo, lethally irradiated LDLR−/− mice which received high fat diet (HFD) for 12 weeks were transplanted with bone marrow from wild type (control) or NPC1 mutant mice, thereby inducing lysosomal cholesterol accumulation in bone marrow cells. The mice were fed a high fat diet low in plant stanol ester levels (HFD) or HFD supplemented with 2% plant stanol esters in the final three weeks of the experiment.

To determine whether plant stanols were able to reduce inflammation in vitro, hepatic inflammation was assessed by cytokine analysis of supernatant and gene expression analysis of inflammatory genes of NPC1 WT and mutant BMDMs incubated with sitostanol or cyclodextrin (carrier control). To determine whether the cholesterol trafficking is affected within the macrophages, gene expression of cholesterol trafficking genes was measured. We found that the administration of plant stanols reduced the inflammatory response by reducing lysosomal cholesterol accumulation within the macrophages.

To determine whether plant stanols improve the pathological liver phenotype in a NPC1 disease-like model, hepatic inflammation in vivo was assessed by gene expression analysis of inflammatory genes and histological staining of KCs and hepatic neutrophils and T-cells after three weeks of stanol ester poor HFD or HFD+2 plant stanol esters. To determine the effect of plant stanols on hepatic lipid metabolism and steatosis, cholesterol, cholesterol precursors and degradation products were analyzed by HPLC and hepatic steatosis was scored in H&E coupes. We found that plant stanols reduced hepatic inflammation and steatosis in a NPC1 disease-like model by reducing lysosomal cholesterol accumulation.

More specifically; we found that plant stanols could reduce inflammation in vitro. Bone marrow derived macrophages (BMDMs) derived from NPC1 mutant and WT mice were stimulated with oxLDL and LPS to induce lysosomal cholesterol accumulation and inflammation in vitro, respectively. By treating the BMDMs with plant stanols or cyclodextrin (carrier control), we observed the effects of plant stanols on inflammation independently of changes in lipid concentrations and the presence of hepatocytes. We assessed TNFα secretion and gene expression of pro-inflammatory mediators. TNFα secretion in vitro was significantly decreased in carrier control- and sitostanol-treated NPC1mut BMDMs compared to carrier control- and sitostanol-treated NPC1WT BMDMs, respectively (p=0.000 and p=0.009, respectively) (FIG. 1A).

Plant sitostanol treatment significantly reduced TNFα secretion in NPC1 WT and mutant BMDMs compared to carrier control-treated NPC1 WT and mutant BMDMs (p=0.000 and p=0.000, respectively) (FIG. 1A). However, when looking to the gene expression of Tnfα merely a trend towards reduced expression was observed in sitostanol-treated NPC1mut BMDMs compared to carrier control-treated NPC1mut BMDMs (p=0.136) (FIG. 1B).

The gene expression of the M2 macrophage phenotype marker, arginase 1, was significantly increased after sitostanol treatment in NPC1mut BMDMs compared to carrier control-treated NPC1mut BMDMs (p=0.000) (FIG. 1B). When looking more into detail at the iNOS/arginase 1 gene expression ratio (a measure for inflammatory status of the macrophages), we observed a trend towards a reduced iNOS/arginase 1 gene expression ratio in sitostanol-treated NPC1mut BMDMs compared to control-treated NPC1mut BMDMs and sitostanol-treated NPC1WT BMDMs (p=0.074 and p=0.124, respectively) (FIG. 1B).

The gene expression of the anti-inflammatory cytokine IL-10 was increased after plant sitostanol treatment in NPC1WT BMDMs (p=0.028) (FIG. 1B). Furthermore, in sitostanol-treated NPC1mut BMDMs a trend was observed towards reduced IL-10 gene expression compared to sitostanol-treated NPC1WT BMDMs (p=0.05) (FIG. 1B). Altogether, these data provide strong evidence for the anti-inflammatory role of sitostanols in NPC1 mutant BMDMs.

We also show herein that plant stanols have an effect on the health condition of NPC1 disease-like mice. Therefore, total body weight and relative body weight of NPC1 WT or mutant bone marrow transplanted LDLR−/− mice was determined nine weeks after marrow transplantation (t=0), after 12 weeks of HFD (t=12) and after 3 weeks of HFD supplemented with 2% plant stanols esters or control diet (plant stanol ester poor HFD) (t=15). Total and relative body weight gain were significantly lower in NPC1mut LDLR−/− mice that were administered the stanol ester diet compared to NPC1WT LDLR−/− mice administered the stanol ester diet (t=15) (FIGS. 2A and 2B).

Furthermore, relative liver and spleen weight were determined after 3 weeks of HFD supplemented with or without 2% stanol esters. Relative liver weight was significantly higher in control- and stanol-treated NPC1mut LDLR−/− mice compared to control- and stanol-treated NPC1WT LDLR−/− mice, respectively (p=0.000 and p=0.014; FIG. 2C).

Moreover, relative liver weight was found to be reduced in stanol-treated NPC1 wt-tp and NPC1mut-tp LDLR−/− mice compared to control-treated NPC1 WT and mutant LDLR−/− mice (p=0.013 and p=0.000, respectively) (FIG. 2C). Relative spleen weight was significantly higher in control- and stanol-treated NPC1mut LDLR−/− mice compared to control- and stanol-treated NPC1WT LDLR−/− mice (p=0.000 and p=0.001, respectively) (FIG. 2D).

We also found that the dietary intake of plant stanols had an impact on on systemic inflammation in NPC1 disease-like mice. For that, the amount of immune cells was measured after 12 weeks of HFD (t=12) followed by 3 weeks of HFD supplemented with 2% plant stanol esters or control diet (t=15) and relative T15/T12 immune counts were calculated. Relative leukocyte counts were significantly increased in control-treated NPC1mut LDLR−/− mice compared to NPC1WT LDLR−/− mice (p=0.023) (FIG. 3A). Surprisingly, supplementation of plant stanols to the HFD significantly reduced the relative leukocyte counts in NPC1mut LDLR−/− mice (p=0.040) (FIG. 3A).

Relative granulocyte counts showed a trend towards reduced counts in stanol-treated compared control-treated NPC1mut LDLR−/− mice (p=0.121) (FIG. 3B).

Looking more into detail to the lymphocytes (NK-, B- and T-cells), relative NK-cell counts showed a trend towards increased counts in control treated NPC1mut LDLR−/− mice compared to NPC1WT LDLR−/− mice (p=0.065) (FIG. 3D). Surprisingly, supplementation of plant stanols to the HFD resulted in a trend towards reduced relative NK-cell counts in NPC1mut LDLR−/− mice (p=0.086) (FIG. 3D).

Relative B-cells counts showed a trend towards increased counts in control-treated NPC1mut LDLR−/− mice compared to NPC1WT LDLR−/− mice (p=0.076) (FIG. 3C). Supplementation of plant stanols to the HFD resulted in a trend to reduced relative B-cells counts in NPC1mut LDLR−/− mice (p=0.111) (FIG. 3C).

Relative T-cell counts were significantly increased in control-treated NPC1mut LDLR−/− mice compared to NPC1WT LDLR−/− mice (p=0.006) (FIG. 3E). Surprisingly, supplementation of plant stanols to the HFD significantly reduced the relative T-cell counts in NPC1mut LDLR−/− mice (p=0.007) (FIG. 3E).

When looking more into dept at the different T-cell subpopulations, relative counts of the CD4+ T-cell subpopulation did not significantly differ between the groups (FIG. 3F). However, relative CD8+ T-cell and Tmemory cell counts were significantly increased in control-treated NPC1mut LDLR−/− mice compared to NPC1WT LDLR−/− mice (FIGS. 3G and 3H). Supplementation of plant stanols to the HFD significantly reduced the relative CD8+ T-cell and Tmemory cell counts in NPC1mut LDLR−/− mice (FIGS. 3G and H).

Overall, these data indicate that Npc1mut-tp Ldlr−/− mice on a HFD show increased systemic inflammation and, more importantly, that supplementation of stanols to the diet of these mice, reduces systemic inflammation.

We also observed that plant stanol ester supplementation affected the plasma lipid, plasma triglyceride and cholesterol levels. These levels were determined nine weeks after marrow transplantation (t=0), after 12 weeks of HFD (t=12) followed by 3 weeks of HFD supplemented with 2% plant stanols esters or control diet (t=15).

After feeding the mice with a HFD with or without plant stanol esters (t=15), plasma triglycerides and cholesterol levels were significantly decreased in stanol-treated NPC1WT LDLR−/− mice compared to control-treated NPC1WT LDLR−/− mice (p=0.007 and p=0.000, respectively) (FIGS. 4A and 4B).

Moreover, plasma triglycerides and cholesterol levels were significantly reduced in control- and stanol-treated NPC1mut LDLR−/− mice compared to control- and stanol-treated NPC1WT LDLR−/− mice (p=0.000, 0.018, 0.000 and 0.003, respectively) (FIGS. 4A and 4B). Lastly, plasma cholesterol was significantly reduced in stanol-treated NPC1mut LDLR−/− mice compared to control-treated NPC1mut LDLR−/− mice (p=0.007) (FIG. 4A).

We also found that plant stanol ester supplementation influenced lipid metabolism in the liver. For that purpose, hepatic cholesterol and triglycerides were measured. Feeding Npc1mut-tp Ldlr−/− mice a HFD resulted in elevated levels of liver cholesterol and reduced levels of liver triglycerides compared to Npc1wt-tp Ldlr−/− given the same HFD (FIGS. 5A and 5B).

Surprisingly, supplementation of 2% plant stanol esters to the diet resulted in a significant decrease of liver cholesterol in both genotypes (FIG. 5A), whereas plant stanol ester supplementation did not affect liver triglyceride levels (FIG. 5B).

We also found that plant stanol esters had an effect on hepatic inflammation. To that effect, we performed, immunohistochemical stainings for the inflammatory cell markers Mac-1 (infiltrated macrophages and neutrophils), CD3 (T cells) and CD68 (resident monocytes/macrophages) on liver sections of Npc1mut-tp Ldlr−/− mice.

Surprisingly, quantification of all immunohistochemical staining revealed increased hepatic inflammation in Npc1mut-tp Ldlr−/− mice compared to Npc1wt-tp Ldlr−/− mice (FIGS. 6A, 6B and 6C). Strikingly, upon supplementation of plant stanol esters to the diet, levels of infiltrating macrophages and neutrophils (FIG. 6A), T cells (FIG. 6B) and resident monocytes/macrophages reduced significantly, indicating plant stanol esters being able to reduce hepatic inflammation.

To confirm these findings at RNA level, hepatic gene expression analysis was performed for the pro-inflammatory markers Tnfα, Ccl2, Caspase1 and Cd68. Completely in line with the histological findings, gene expression levels of all inflammatory markers increased in Npc1mut-tp Ldlr−/− mice compared to Npc1wt-tp Ldlr−/− mice (FIGS. 7A-7D). Similarly, plant stanol supplementation reduced expression levels of the pro-inflammatory markers (FIGS. 7A-7D).

Taken together, these findings prove that plant stanol esters can act as anti-inflammatory agents and that they can overcome lysosomal cholesterol-induced hepatic inflammation.

The invention therefore relates to a composition comprising a plant sterol or plant stanol for use in the treatment of Niemann-Pick disease type C1. This may also be worded as a method for treating Niemann-Pick disease type C1, wherein a composition comprising a plant sterol or plant stanol is administered to a subject in need of such a treatment. In yet other words, the invention relates to the use of a plant sterol or plant stanol for the manufacture of a medicament for the treatment of Niemann-Pick disease type C1.

Phytosterols, which encompass plant sterols and stanols, are steroid compounds similar to cholesterol which occur in plants and vary only in carbon side chains and/or presence or absence of a double bond. StenoIs are saturated sterols, having no double bonds in the sterol ring structure. More than 200 sterols and related compounds have been identified.

In a preferred embodiment, the invention relates to a composition for use as described above, wherein the plant stanol is a chemically saturated plant sterol.

In another embodiment, the invention relates to a composition for use as described herein, wherein the plant stanol is esterified with a fatty acid to form a fatty acid ester.

A stanol ester is a heterogeneous group of chemical compounds known to reduce the level of low-density lipoprotein (LDL) cholesterol in blood when ingested, though to a much lesser degree than prescription drugs such as statins. The starting material is phytosterols from plants. These are first hydrogenated to give a plant stanol which is then esterified with a mixture of fatty acids also derived from plants. Plant stanol esters are also found naturally occurring in small quantities in fruits, vegetables, nuts, seeds, cereals, legumes, and vegetable oils.

Stanol ester is often added to rapeseed oil based margarine or other foods for its health benefits. Studies have indicated that consumption of about 2-3 grams per day provides a reduction in LDL cholesterol of about 10-15%. The compound itself passes through the gut without entering the blood stream or lymph. Its presence, however, reduces both the amount of cholesterol the body absorbs from food and the reabsorption of the cholesterol component of bile.

Sterol esters can also be used for the same purpose. These compounds have the same effect to LDL, but they are partially absorbed by the body.

The plant stanols and sterols described herein may consist or comprise a mixture of different sterols and stanols.

The richest naturally occurring sources of phytosterols are vegetable oils and products made from them. They can be present in the free form and as esters of fatty acid/cinnamic acid or as glycosides, respectively. The bound form is usually hydrolyzed in the small intestines by pancreatic enzymes. Nuts, which are rich in phytosterols, are often eaten in smaller amounts, but can still significantly contribute to total phytosterol intake. Cereal products, vegetables, fruit and berries, which are not as rich in phytosterols, may also be significant sources of phytosterols due to their higher intakes. The intake of naturally occurring phytosterols ranges between about 150-450 mg/day depending on eating habits. Specially designed vegetarian experimental diets have been produced yielding upwards of 700 mg/day. The most commonly occurring phytosterols in the human diet are β-sitosterol, campesterol and stigmasterol, which account for about 65%, 30% and 3% of diet contents, respectively. The most common plant stanols in the human diet are sitostanol and campestanol, which combined make up about 5% of dietary phytosterol.

Hence, the invention also relates to a composition for use as described herein wherein the composition comprises a mixture comprising sitostanol and campestanol, such as for instance a mixture comprising about 70% sitostanol and about 30% campestanol.

The molecular formula for some sterols and stanols are reproduced herein below.

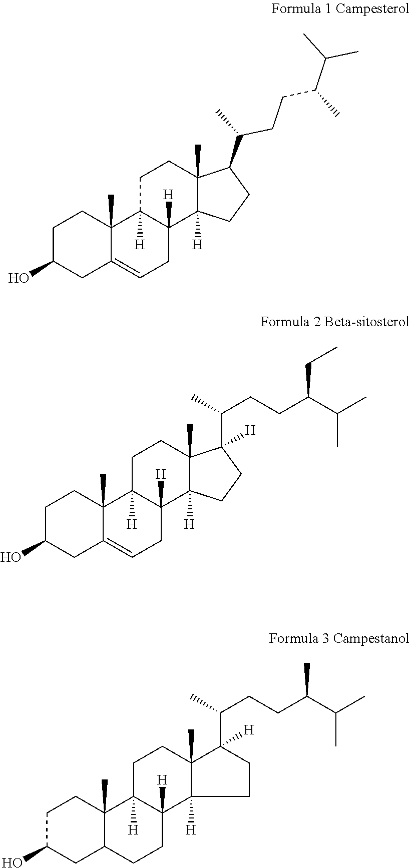

Using his general knowledge and the information provided herein, the skilled person will be able to establish the optimal dose for the desired effect in an organism of his choice. If the organism to be treated is a human, the optimal dose may be in the range of 3-9 grams of sterols and/or stanols per person per day. This is to be interpreted as that the sum of the amount of plant sterols and plant stanols that is to be administered to a human Is preferably between 3 and 9 grams per day. Studies have shown that such is a safe dose and results in plasma concentrations that are generally considered as safe.

Whereas a normal control diet in humans yielded a plasma concentration of 4.7 microgram per dl, administration of 3 grams of sitostanol per day yielded a plasma concentration of 40.9 microgram per dl and 9 grams per day yielded a plasma concentration of 57.2 microgram per dl.

For campestanol, the values were 4.4 microgram per dl in humans with a control diet, 17.5 microgram per dl for humans that were given 3 grams of campestanol per day and 28.3 micrograms per dl for humans that were given 9 grams of campestanol per day.

### FIGURE LEGENDS

FIG. 1: Inflammatory parameters of WT and NPC1mut BMDMs treated with or without sitostanol. ** Indicates p<0.01 and *** p<0.001 compared to the respective Npc1wt-tp Ldlr−/− mice; ### indicates p<0.001 compared to the respective HFD without stanol-treated mice by use of Two-way ANOVA with Tuckey post-hoc analysis.

FIG. 2: Physiological parameters of NPC1 wt-tp and NPC1mut-tp LDLR−/− mice supplemented with or without 2% plant stanol ester to the HFD diet. * Indicates p<0.05, ** p<0.01 and *** p<0.001 compared to the respective Npc1wt-tp Ldlr−/− mice; # p<0.05 and ### p<0.001 compared to the respective HFD without stanol-treated mice by use of Two-way ANOVA with Tuckey post-hoc analysis.

FIG. 3: Relative difference in leukocytes (A), granulocytes (B), B cells (C), NK cells (D), T cells (E), CD4+ T cells (F), CD8+ T cells (G), T memory cells (H) between T12 and T15. * Indicates p<0.05 and ** p<0.01 compared to the respective Npc1wt-tp Ldlr−/− mice; # p<0.05 and ## p<0.01 compared to the respective HFD without stanol-treated mice by use of Two-way ANOVA with Tuckey post-hoc analysis.

FIG. 4: Plasma lipid levels of Npc1wt-tp and Npc1mut-tp Ldlr−/− mice on HFD supplemented with or without 2% plant stanol esters. * Indicates p<0.05, ** p<0.01 and *** p<0.001 compared to the respective Npc1wt-tp Ldlr−/− mice; ## p<0.01 and ### p<0.001 compared to the respective HFD without stanol-treated mice by use of Two-way ANOVA with Tuckey post-hoc analysis.

FIG. 5: Liver lipid levels of Npc1wt-tp and Npc1mut-tp Ldlr−/− mice on HFD supplemented with or without 2% plant stanol esters. * Indicates p<0.05, ** p<0.01 and *** p<0.001 compared to the respective Npc1wt-tp Ldlr−/− mice; ## p<0.01 compared to the respective HFD without stanol-treated mice by use of Two-way ANOVA with Tuckey post-hoc analysis.

FIG. 6: Quantification of hepatic immunohistochemical stainings of Npc1wt-tp and Npc1mut-tp Ldlr−/− mice on HFD supplemented with or without 2% plant stanol esters. **** Indicates p<0.001 compared to the respective Npc1wt-tp Ldlr−/− mice; # p<0.05, ## p<0.01 and ### p<0.001 compared to the respective HFD without stanol-treated mice by use of Two-way ANOVA with Tuckey post-hoc analysis.

FIG. 7: Hepatic gene expression analysis of inflammatory markers in Npc1wt-tp and Npc1mut-tp Ldlr−/− mice on HFD supplemented with or without 2% plant stanol esters. Data are shown relative to Npc1wt-tp Ldlr−/− mice on HFD without the stanols. * Indicates p 0.05 and *** p<0.001 compared to the respective Npc1wt-tp Ldlr−/− mice; # indicates p 0.05 and ### p<0.001 compared to the respective HFD without stanol-treated mice by use of Two-way ANOVA with Tuckey post-hoc analysis.

FIG. 8: Experimental set-up. First, the mothers of the pups that were going to be included in the experiment received the control/stanol ester diet in the food. As a consequence, in the first 2 weeks of the experiment, the pups received the stanol esters via the breastmilk of the mothers. After two weeks of the experiment, the mothers were separated from the pups. As a results, the pups now continued receiving the diet, but now by means of solid food. After the age of 7 weeks, all mice were sacrificed.

FIG. 9: Total weight development and liver weight. ^{####} Indicates p<0.0001 compared to Wt mice, **** p<0.0001 compared to Npc1^{mut} mice on control diet by use of unpaired t test.

FIG. 10: Hepatic gene expression levels. ^{####} Indicates p<0.0001 compared to Wt mice; * p<0.05, ** p<0.01, *** p<0.001 and **** p<0.0001 compared to Npc1^{mut} mice on control diet by use of unpaired t test.

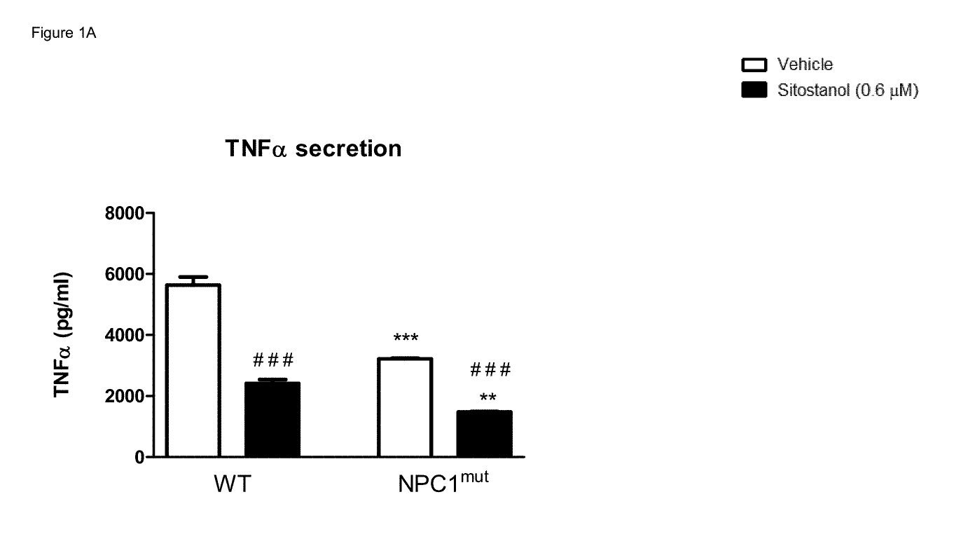

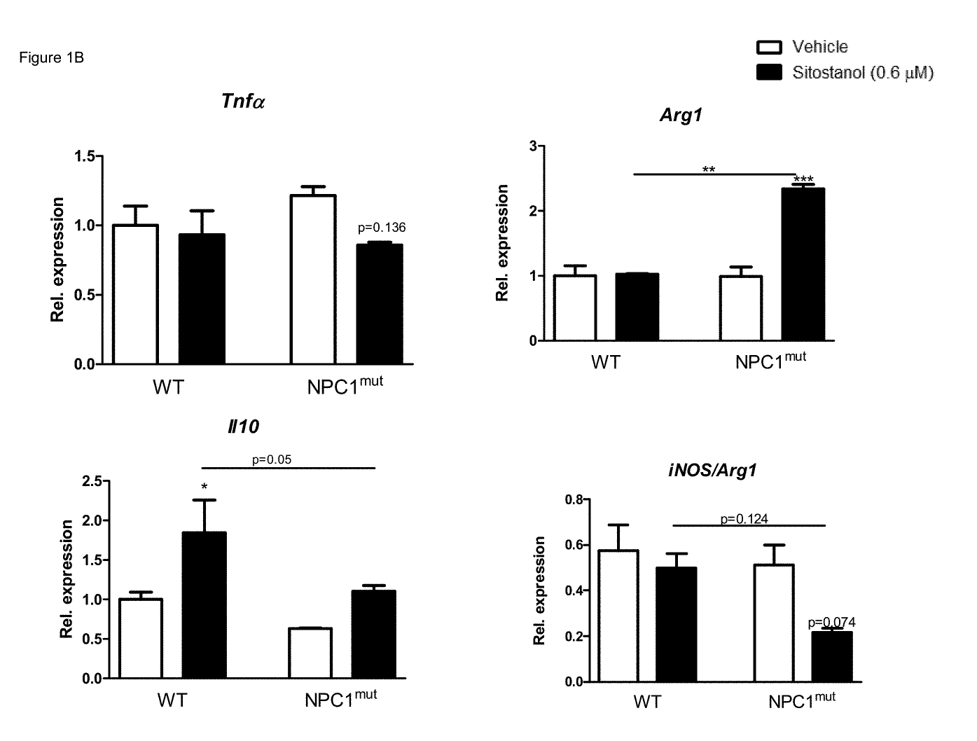

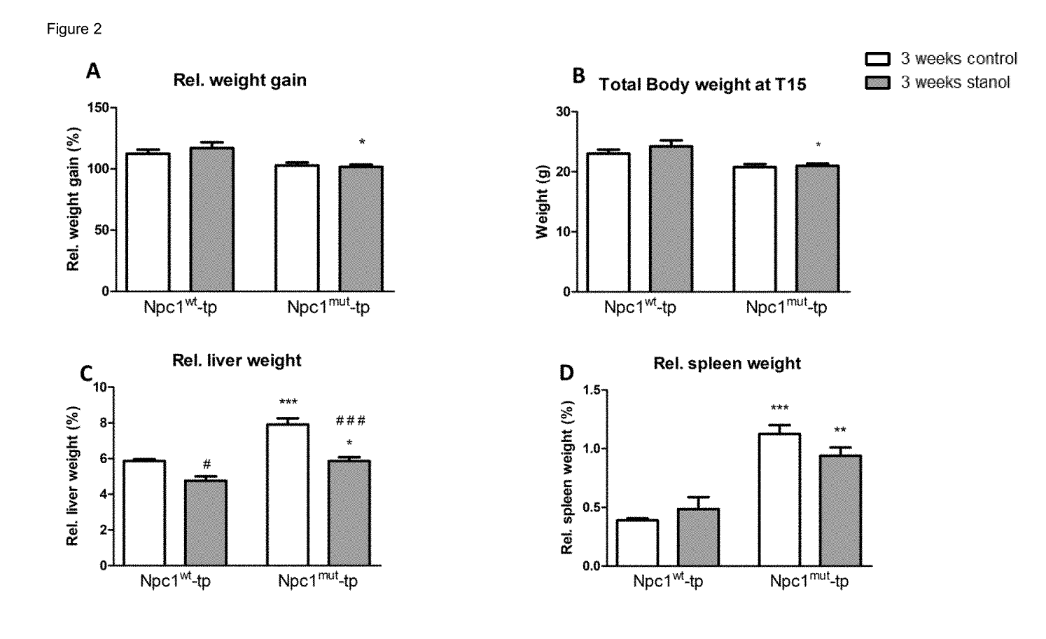

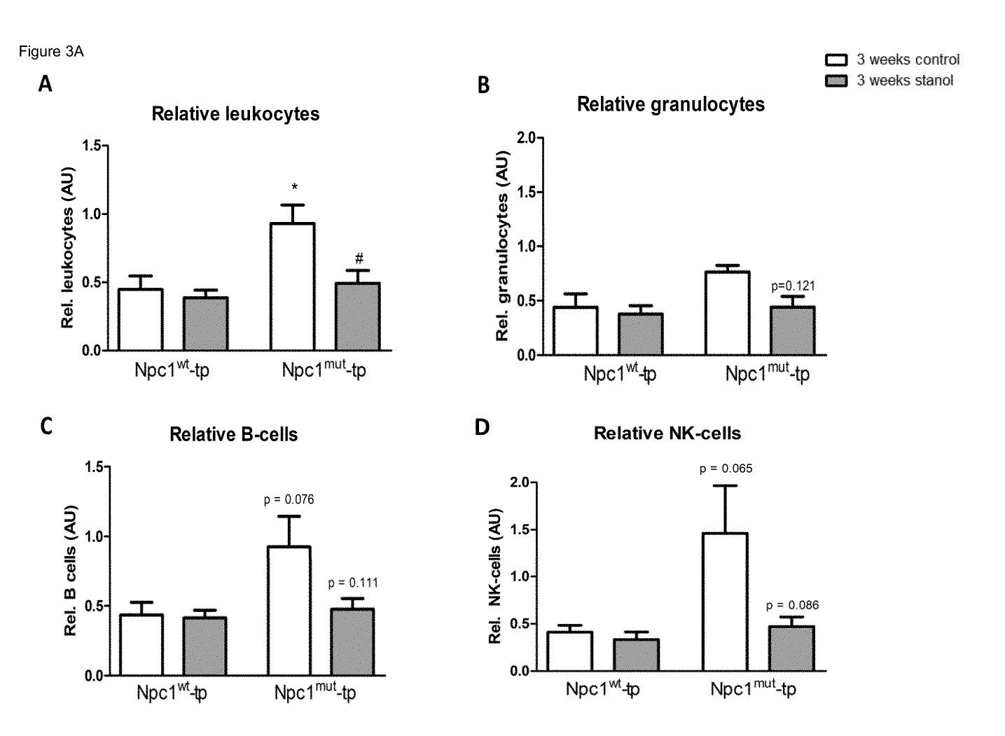

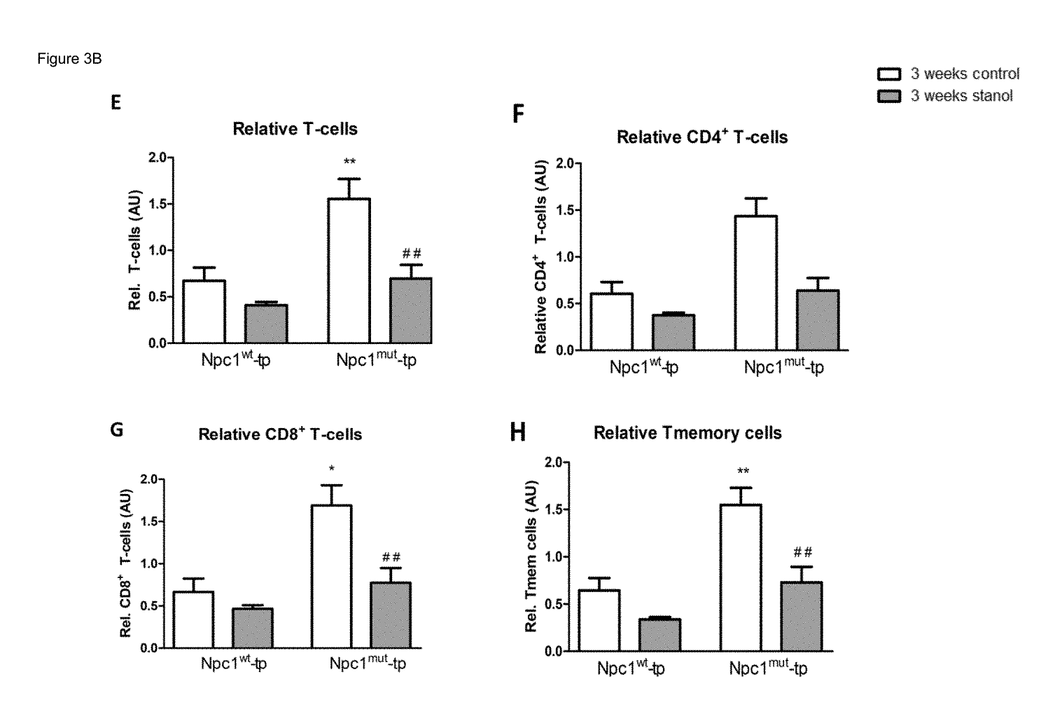

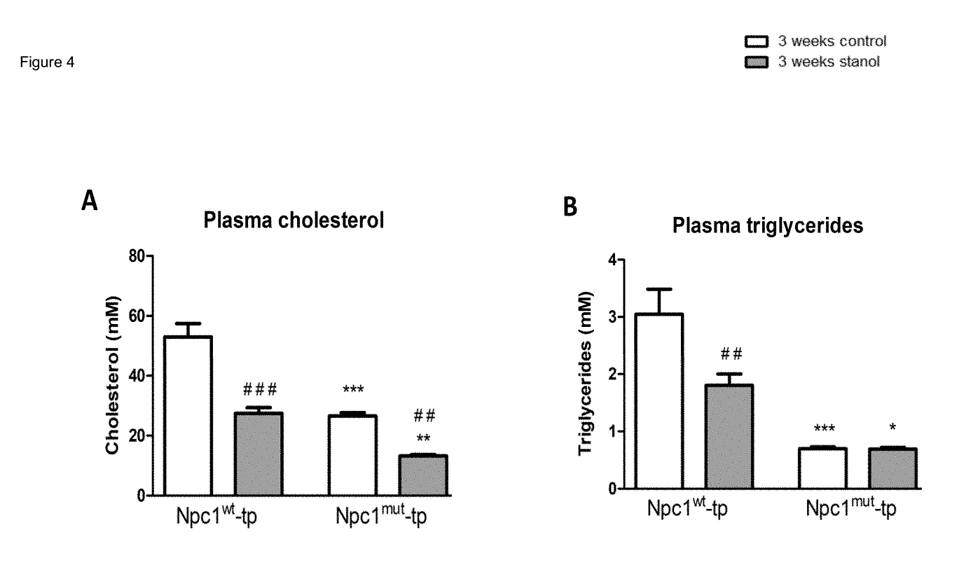

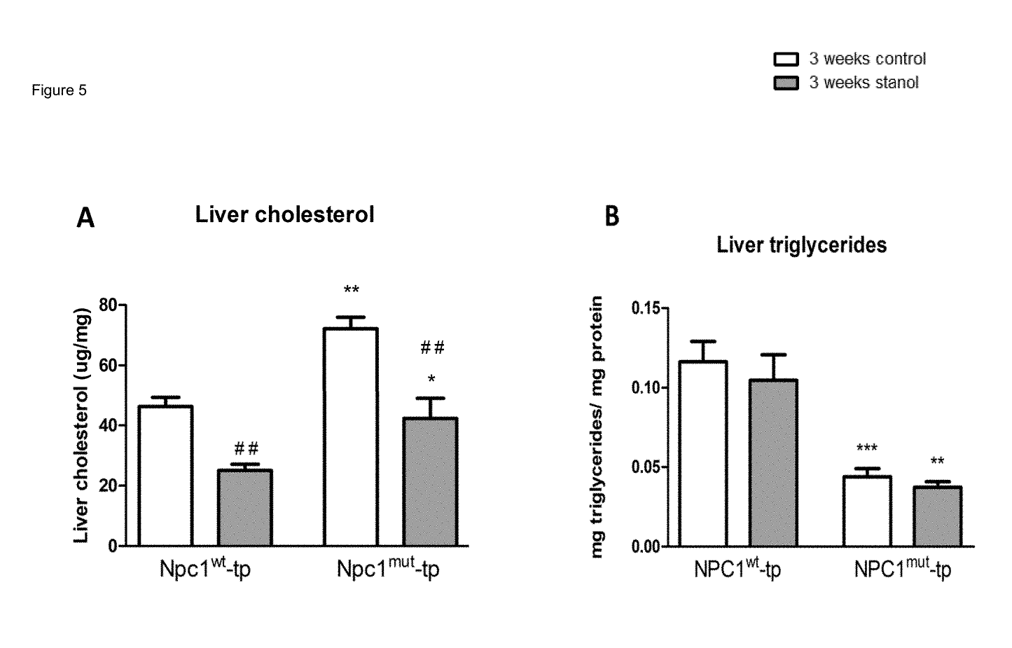

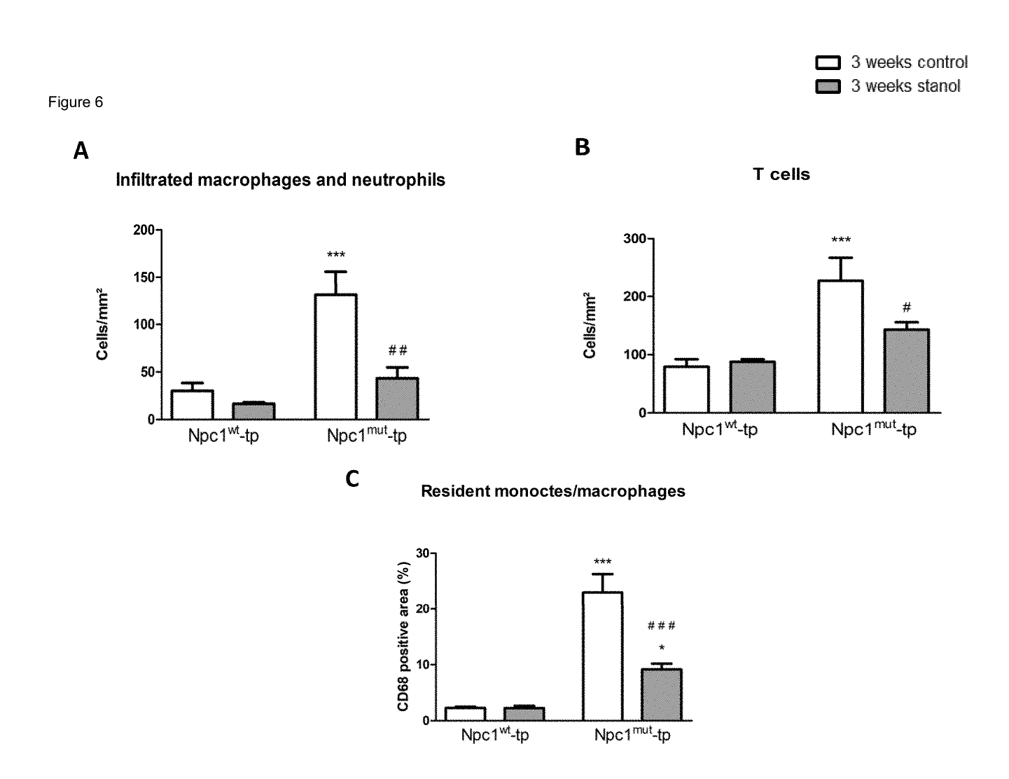

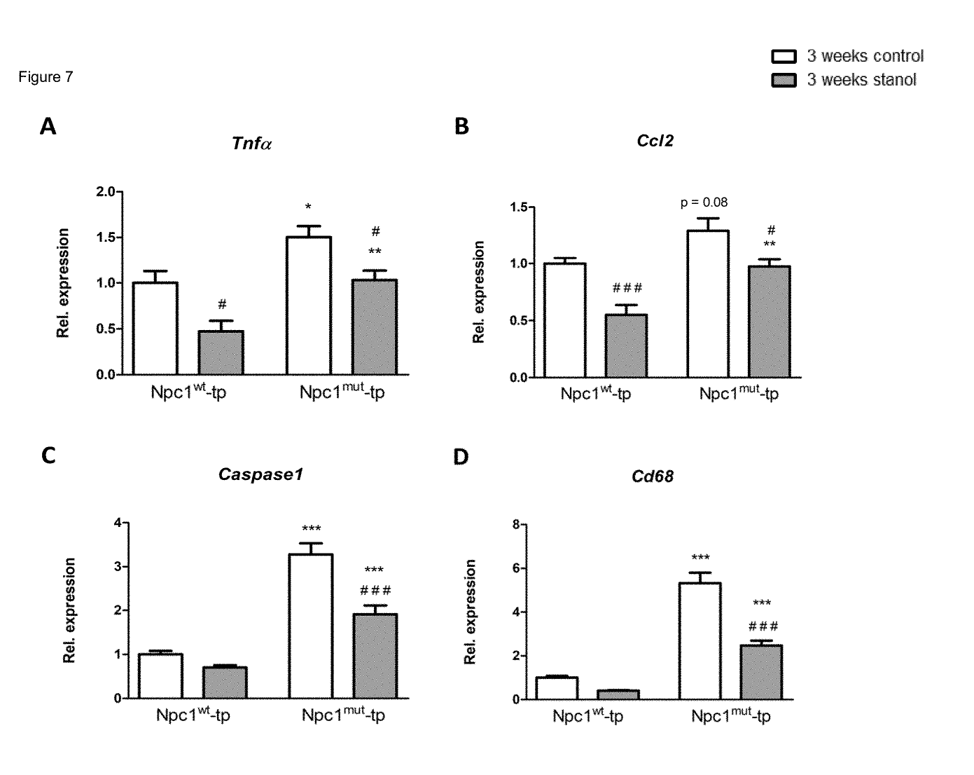

### EXAMPLES

### Example 1: In Vitro Study with Bone Marrow-Derived Macrophages (BMDMs)

Bone-marrow derived cells (BMDCs) were isolated from tibea and femurs of NPC1 wild type (WT) and mutant mice on a C57BL/6 background. BMDCs were cultured for 8-9 days in RPMI-1640 (GIBCO Invitrogen, Breda, The Netherlands) with 10% heat-inactivated fetal calf serum (Bodinco B. V. Alkmaar, The Netherlands), penicillin (100 U/ml), streptomycin (100 μg/ml) and L-glutamine (2 mM) (all GIBCO invitrogen, Breda, The Netherlands) enriched with 20% L929-conditioned medium (LCM) to generate bone marrow-derived macrophages (BMDMs). After attachment, the BMDMs were seeded at 350,000 cells per well in 24 wells plates and incubated with oxLDL for 24. After the oxLDL stimulation, the cells were washed and incubated with cyclodextrin (carrier control) or 0.6 μM sitostanol for 4 hours. The BMDMs were subsequently washed and stimulated with 100 ng/ml LPS for 4 hours to generate an inflammatory response. Finally, BMDMs were lysed for RNA isolation. Gene expression analysis of inflammatory (TNFα, Arginase 1 and inducible NOS), anti-inflammatory (IL-10) and lysosomal cholesterol trafficking (NPC1 and NPC2) genes was performed. Furthermore, the supernatant was frozen until protein expression analysis of the pro-inflammatory TNFα was performed by Enzyme-Linked Immunosorbent Assay (ELISA).

### Example 2: TNFα Enzyme-Linked Immunosorbent Assay (ELISA)

Mouse TNFα secreted protein levels were determined by using the TNFα ELISA kit (mouse TNFα ELISA Ready-SET-Go!, eBioscience, San Diego, Calif.). Briefly, the high affinity protein binding ELISA plate (Nunc Maxisorp, Rochester, N.Y.) was incubated with 1:250 capture antibody in 1× coating buffer overnight at 4° C. The plates were washed with washing buffer (0.05% Tween in 1×PBS) and subsequently incubated with blocking buffer (1:5 Assay Diluent in distilled water) for 1 hour to prevent non-specific binding. After blockage of the plates, the plates were washed and subsequently incubated with the standards and samples for 2 hours at room temperature in the dark. The plates were washed and incubated with 1:250 detection antibody in 1× Assay Diluent for 1 hour at room temperature. After the incubation with the detection antibody, the plates were washed and incubated with 1:250 avidin-horseradish peroxidase (avidin-HRP) in 1× Assay Diluent at room temperature for 30 minutes. The plates were subsequently washed and incubated with the 1×TMB substrate solution at room temperature. After 15 minutes of incubation with the TMB substrate solution, the reaction as stopped by using the stop solution (2M H2SO4). Optical density (OD) was measured at 450 nm by using a spectrophotometer and TNFα concentrations were determined.

### Example 3: Mice, Bone Marrow Transplantation and Diet

To induce a NPC1 disease-like mouse model, 12 week-old female LDLR−/− mice on a C57BL/6 background were exposed to full-body irradiation with a lethal dose of 10 Gy one day before bone marrow isolation. Bone marrow was isolated of NPC1 WT and mutant mice. Lethally irradiated LDLR−/− mice were transplanted with 107 bone marrow cells from NPC1mut or NPC1WT mice by tail vein injection. Chimerism was determined by performing quantitative Polymerase Chain Reaction (qPCR) to calculate the percentage of LDLR−/− DNA (remaining recipient bone marrow) in the blood of the transplanted mice. After a recovery period of 9 weeks, 16 LDLR−/− NPC1mut and 16 LDLR−/− NPC1WT mice received high fat diet (HFD; 60 kcal % fat; D12492, Research Diets, New Brunswick) for 12 weeks. After 12 weeks of HFD, blood was drawn by performing a tail vein punction.

To investigate whether plant stanols improve the pathological liver phenotype in a NCP1 disease-like model in vivo, eight LDLR−/− NPC1mut and eight LDLR−/− NPC1WT mice are assigned to a HFD supplemented with 2% plant stanols esters for three weeks. As a control, eight LDLR−/− NPC1mut and eight LDLR−/− NPC1WT age-matched litter mates received a plant stanols ester poor HFD (control diet) for three weeks. The number of mice assigned to the experimental and control group is based on the sample size calculation of Dupont and Plummer considering a power (β) of 80%, accepted significance level (α) of 0.05, expected effect size (μ1-μ2) of 25% and variation coefficient of 13%. After 15 weeks of diet, experimental and control LDLR−/− NPC1 mutant and WT mice were sacrificed by CO2 asphyxiation. The liver and spleen were weighted and were subsequently snap frozen in liquid nitrogen and stored at −80 degrees or fixed in 4% formaldehyde/PBS. Blood was drawn by performing a heart puncture.

### Example 4: Fluorescence-Activated Cell Sorting (FACS)

Blood collected by tail vein function and heart puncture after 12 weeks of HFD (or t=12) and after 3 weeks of HFD supplemented with 2% plant stanols esters or plant stanol ester poor HFD (or t=15), respectively, was added to PBS containing EDTA to prevent blood clotting. According to the TruDifCount protocol, the absolute counts of total leukocytes, non-TB cells, T-cells, Thelper cells, cytotoxic T-cells, memory cytotoxic T-cells, B-cells, Non-Natural killer cells (non-NK cells) NK-cells, NKT-cells, granulocytes, monocytes, Ly6C positive and negative cells, T-cells (NKT-cells) were determined by FACS analysis. Briefly, 1:100 FCR block (14-0161, eBioscience, USA) was added to the blood to block non-specific Fc receptor mediated antibody binding. After 10 minutes of incubation in the dark, TRuDifBlood antibody mix was added to detect the immune cell populations in the blood. After 20 minutes incubation in the dark, erylysis solution was added to remove the erythrocytes. After 15 minutes of incubation in the dark, the immune cell populations were measured BD FACSCanto II (bdbiosicience, Belgium).

### Example 5: Hepatic Immunohistochemical Stainings of the Liver

Frozen 7 μm liver sections of stanol- and control treated NPC1 WT and mutant LDLR−/− mice were prepared by using a microtome (−20° C., Microm HM 560) and were fixed in dry acetone. The liver sections were blocked for endogenous peroxidase by incubation with 0.03% H2O2 for 5 minutes. Prior to the incubation with the primary antibody, the liver sections were blocked for endogenous biotin by incubation with 1:5 red kit avidin D (ABC kit, Vector Laboratories, USA) in 4% fetal calf serum (FCS) and 1× phosphate buffered saline for 30 minutes. Subsequently, the liver sections were blocked for endogenous avidin by incubation with 1:5 red kit biotin (ABC kit, Vector Laboratories, USA) in 4% FCS, 1×PBS and the primary antibody for 60 minutes. Primary antibodies were directed against infiltrated macrophages and neutrophils (1:500 rat-anti-mouse MAC1, clone M1/70), resident Kupffer cells (1:100 rat-anti-mouse CD68, clone FA11) and T-cells (1:20 rat-anti-mouse CD3). As a secondary antibody anti-rat-BIO (1:200), dissolved in 4% FCS, 2% normal mouse serum (NMS) and 1×PBS, was used. After incubation with the secondary antibody, liver section were washed and incubated with the ABC kit to amplify the signal. As a colour substrate 3-amino-9-ethylcarbazole (ACE) (A85SK-4200.S1; Bio-connect, Huissen, The Netherlands) was used. For nuclear counterstaining, Haematoxylin (H; 4085.9002, Klinipath, Duiven, The Netherlands) was used. By using faramount aqueous mounting medium (S302580; DAKO, Glostrup, Denmark), the liver sections were covered with a cover slip. Microscopical pictures of the liver sections were taken by using a Nikon digital camera DMX1200 and ACT-1 v2.63 software (Nikon Instruments Europe, Amstelveen, The Netherlands). Immune cells were counted in six microscopical views (original magnification, 200×) and were indicated as number of cells per square millimetre.

### Example 6: Hematoxylin and Eosin (HE) Staining of the Liver

Paraffin-embedded 4 μm liver sections were prepared by a microtome (Leica Reichert-Jung Biocut, Rijswijk). The liver sections were stained with haematoxylin (H; 4085.9002; Klinipath, Duiven, The Netherlands) and Eosin (E; E4382; Sigma-Aldrich) (supplement 7.3). Microscopical pictures (original magnification, 200×) were taken with a Nikon digital camera DMX1200 and ACT-1 v2.63 software (Nikon Corporation, Tokyo, Japan). The presence of hepatic steatosis and inflammation were scored and a high score indicated a high level of hepatic steatosis and inflammation, respectively.

### Example 7: RNA Isolation

Total RNA was isolated from frozen liver tissue of stanol- and control treated NPC1 WT and mutant LDLR−/− mice. Briefly, the liver tissues were homogenized in TriReagent by using the MiniBeadBeater (3110BXEUR, Biospec Products, Bartlesville, USA). The homogenate was centrifuged to remove insoluble materials and subsequently incubated at room temperature for 5 minutes to ensure complete dissociation of the nucleoprotein complex. Chloroform was added to induce three phases: the red organic phase containing protein, the interphase containing DNA and the colorless aqueous upper phase containing RNA. The aqueous upper phase was transferred to a new tube and isopropanol was added. After centrifugation, the RNA precipitated. The supernatant was removed and the resultant RNA pellet was washed with 70% ethanol. The RNA pellet was dried and subsequently resuspended in autocleaved miliQ. By using Nanodrop ND-1000 spectrometer, the quality and quantity of the RNA was determined (Witec AG, Lucerne, Switzerland). All materials used were RNA free and samples were placed on ice during the RNA isolation procedure.

### Example 8: Quantitative Polymerase Chain Reaction (qPCR)

Total RNA (500 ng) of the liver was reverse transcribed in first-strand complementary DNA (cDNA) by using the iScript cDNA synthesis kit (170-8891; Bio-Rad, Hercules, USA) according to the manufacturer's instructions. Changes in the expression of inflammatory, lysosomal cholesterol trafficking, lysosome-associated, apoptotic and lipid transport genes were determined by qPCR of 10 ng cDNA on Bio-Rad MyIQ with IQ5 v2.1 software (Applied Biosystems ABI7900) using IQ SensiMix SYBR master mix with fluorescein (Bioline, London, UK). Cyclophillin A (Ppia), Ribosomal protein S12 and beta-actin were used as reference genes to standardize for the amount of cDNA. By using default settings in primer Express version 2.0 (Applied Biosystems, Forster City, Calif., USA), primer sets for the selected genes were developed.

### Example 9: Liver Lipid Levels

Hepatic cholesterol and precursors were analyzed by High-performance liquid chromatography. The isocratic HPLC system (VWR, Darmstadt, Germany) was composed of separate Hitachi modules with an in-line vacuum degasser, a L-2130 pump, a L-2200 autosampler, a L-2300 column oven and a L-2485 fluorescence detector which were all under the control of Elite LaChrom Software V.3.1.7. A nucleodur C18 Gravity column was used for separation. For the separation of the lipid compounds, solid phase extraction vacuum manifold was used. All solvent were HPLC grade (LiChrosolv, VWR, Darmstadt, Germany) and disposable SPE cardridge (MAcherey-Nagel, Duren, Germany) were used.

### Example 10: Statistical Analysis

The data were statistically analyzed by one-way analysis of two-way variance (ANOVA) with Tukey's Multiple Comparison host-hoc test for comparing multiple conditions using IBM SPSS Statistics 2.1 (IBM, Amsterdam, The Netherlands). Data were expressed as mean±standard error of mean (SEM) and considered as significant at P<0.05 (*P<0.05, **P<0.01 and ***P<0.001, respectively).

### Example 11: Therapeutic Potential of Sitostanols in Niemann-Pick Type C1 Disease

In this experiment, the therapeutical potential of sitostanols in Niemann-Pick type C1 disease (NPC1) was further investigated. Two week-old Niemann-Pick type C1^{nih} mutant (Npc1^{mut}) mice received a chow diet enriched with 2 or 6% (w/w) plant stanol esters. As controls, an experimental group of wildtype, healthy mice and Npc1^{mut} mice were given a plant stanol ester poor chow diet. The plant stanol esters used are a mixture of sitostanol and campestanol (85:15 ratio).

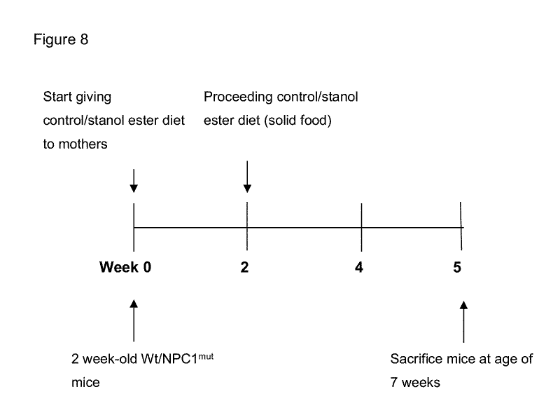

Furthermore, in the first two weeks (week 0 until 2), the two week-old mice received the control/stanol ester diet via the breastmilk of the mother. Afterwards (week 0 until 5), the mice received the control/stanol ester diet via solid food. A more detailed explanation of the experimental set-up can also be found in FIG. 8.

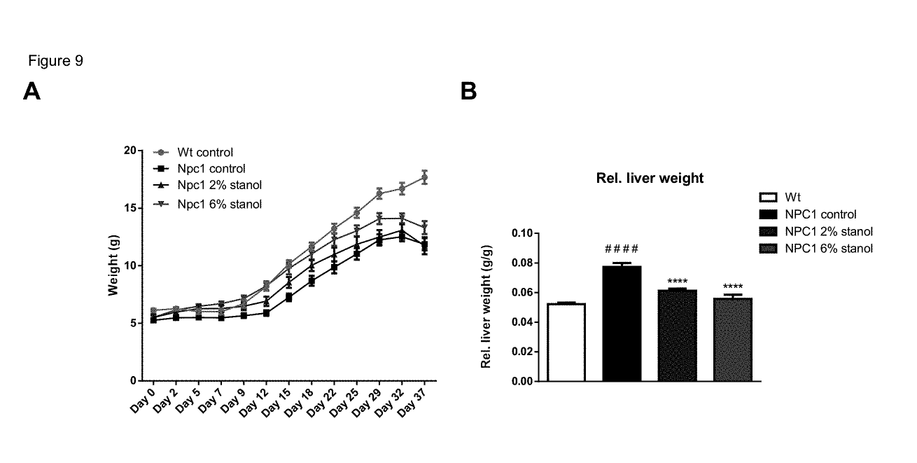

To retrieve an overall view of the health status of the mice, total weight development and relative liver weight were assessed. For the total liver weight (FIG. 9A), Npc1^{mut} mice that received the control diet showed a delay in weight gain compared to Wt mice, confirming the pathological phenotype of NPC1 (FIG. 9A). Relevantly, Npc1^{mut} mice that received chow diet supplemented with 2 or 6% stanol esters demonstrated an improved weight development compared to Npc1^{mut} mice on control diet, suggesting an improvement in phenotype after stanol ester administration (FIG. 9A). Next, while relative liver weight increased in Npc1^{mut} mice on control diet, supplementation of 2 or 6% stanols dramatically reduced liver weight, suggesting an improvement in hepatic physiology (FIG. 9B).

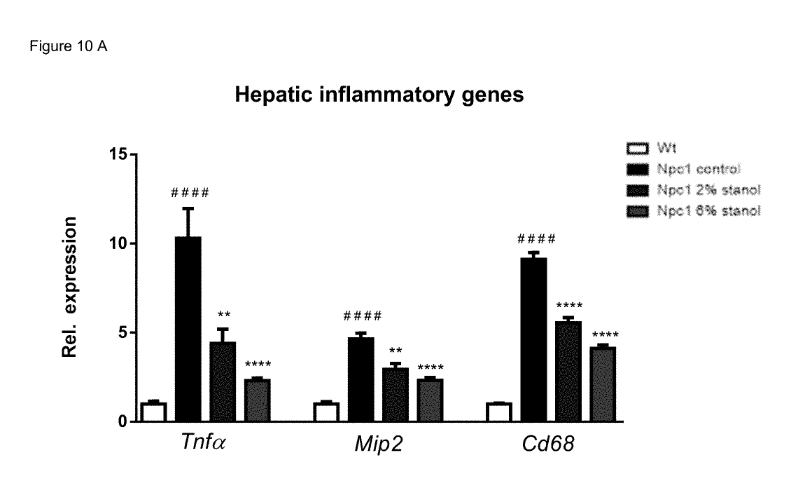

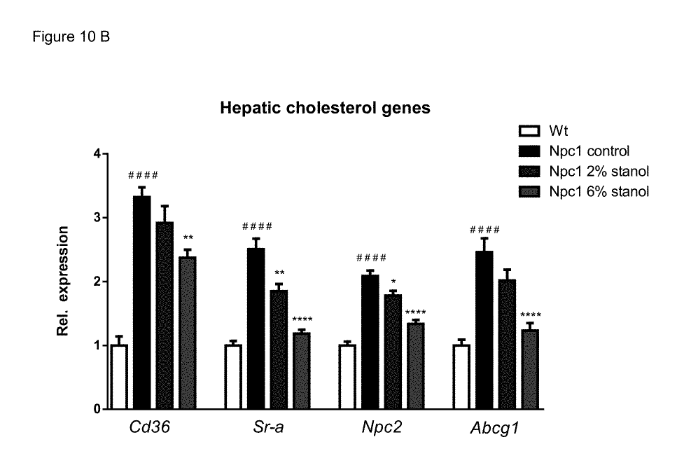

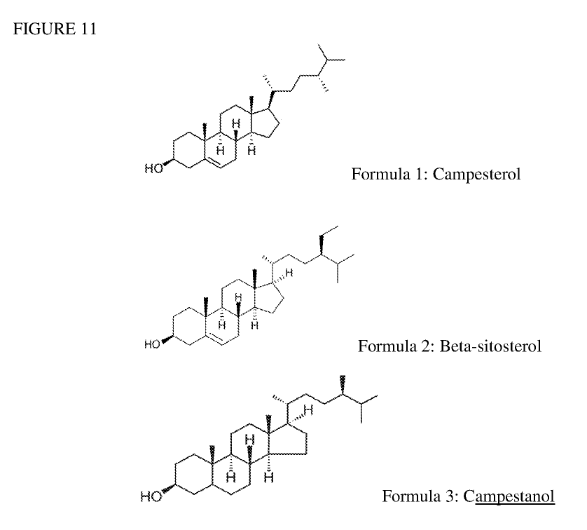

To gain additional insight into the hepatic physiology of Npc1^{mut} mice on control/stanol ester diet, we performed hepatic gene expression analysis of inflammatory (FIG. 10A) and cholesterol (FIG. 10B) markers. The inflammatory markers Tnfα, Mip2 and Cd68 increased in Npc1^{mut} mice on control diet compared to Wt mice.

Supplementation of the diet with stanol esters resulted in reduced expression levels of each inflammatory gene, indicating a dramatic improvement in hepatic inflammation (FIG. 10A). Furthermore, also cholesterol metabolism improved after supplementation of stanol esters to the diet (FIG. 10B). Specifically, while the expression levels of scavenger receptors Cd36 and Sr-a increased in Npc1^{mut} mice on control diet, supplementation of stanol esters (both 2 and 6%) reduced expression levels dramatically (FIG. 10B). Also intralysosomal lipids (indicated by Npc2) and cholesterol efflux (indicated by Abcg1) appeared to show the same trends, indicating an improved cholesterol metabolism after supplementation of stanol esters to Npc1^{mut} mice.

### REFERENCES

1. Lyseng-Williamson K A. Miglustat: a review of its use in Niemann-Pick disease type C. Drugs. 2014; 74(1):61-74.
2. Abi-Mosleh L, Infante R E, Radhakrishnan A, Goldstein J L, Brown M S. Cyclodextrin overcomes deficient lysosome-to-endoplasmic reticulum transport of cholesterol in Niemann-Pick type C cells. Proceedings of the National Academy of Sciences of the United States of America. 2009; 106(46):19316-21.
3. Rosenbaum A I, Zhang G, Warren J D, Maxfield F R. Endocytosis of beta-cyclodextrins is responsible for cholesterol reduction in Niemann-Pick type C mutant cells. Proceedings of the National Academy of Sciences of the United States of America. 2010; 107(12):5477-82.
4. Pontikis C C, Davidson C D, Walkley S U, Platt F M, Begley D J. Cyclodextrin alleviates neuronal storage of cholesterol in Niemann-Pick C disease without evidence of detectable blood-brain barrier permeability. Journal of inherited metabolic disease. 2013; 36(3):491-8.

1. A composition comprising a plant sterol or plant stanol or an ester thereof for use in the treatment of Niemann-Pick disease type C1
wherein the plant stanol is a mixture; and
wherein the mixture comprises mixture of sitostanol and campestanol.

2.-5. (canceled)

6. The composition for use according to claim 1, wherein the mixture consists of about 70% sitostanol and about 30% campestanol.

7. (canceled)
